(19) United States
(12) Patent Application Publication
(10) Pub. No.: US 2003/0176343 A1
(43) Pub. Date: Sep. 18, 2003

(54) Diagnostics and therapeutic uses of topors

(75) Inventors: Eric H. Rubin, Belle Mead, NJ (US); Ahamed Saleem, Highland Park, NJ (US); Zeshaan Rasheed, Piscataway, NJ (US); Paul Haluska JR., Rochester, MN (US)

Correspondence Address: PERKINS COIE LLP, POST OFFICE BOX 1208, SEATTLE, WA, 98111-1208, US

(73) Assignee: UNIVERSITY OF MEDICINE AND DENTISTRY OF NEW JERSEY

(21) Appl. No.: 10/339,924

(22) Filed: Jan. 9, 2003

Related U.S. Application Data
(60) Provisional application No. 60/346,953, filed on Jan. 9, 2002.

(51) Int. Cl. C12Q 1/68; G01N 33/574; A61K 48/00; A61K 38/17; C07K 16/40

(52) U.S. Cl. 514/12; 514/44; 435/6; 435/7.23; 530/388.26; 424/93.2

(57) ABSTRACT

Topors and topors antibody can be used to manipulate the presence and function of both Top1 and p53 in cells, thus controlling the function of the Top1 and p53 proteins. Topors is implicated in prevention of tumorigenesis through its role in DNA repair and preventing faulty or mutated DNA from replicating. Topors can be used therapeutically as a medicament and topors DNA can be used in gene therapy. Topors antibody may be used to detect the presence of cancer by screening for the absence of topors in a given cell or tissue sample. Kits comprising the topors antibody are also contemplated.

### CROSS-REFERENCE TO RELATED APPLICATIONS

The present utility patent application claims priority to provisional patent application 60/346,953 (Rubin, et al.), filed Jan. 9, 2002, which is incorporated by reference in its entirety herein.

### GOVERNMENT INTEREST

This research is in part funded by National Institutes of Health grant number GM59170. The government may own certain rights in the present invention.

### FIELD OF THE INVENTION

The present invention relates to the field of products and methods for treatment of diseases relating to uncontrolled DNA replication and/or uncontrolled proliferation of cells. The present invention particularly relates to the use of topors and topors antibody in various diagnostic and therapeutic manners for preventing and treating cancer and cellular stress-related diseases.

### BACKGROUND

Various publications or patents may be mentioned throughout this application or at the end of the specification to describe the state of the art to which the invention pertains. Each of these references is incorporated by reference herein. Complete citations of scientific publications are set forth at the end of the specification.

Topoisomerase 1 is a DNA binding protein that regulates DNA topology through changing the degree of supercoiling by cutting the DNA strand. Human DNA top1 is a 100 kDa nuclear protein and is the target of an important class of antineoplastic agents called camptothecins (CPTs). CPTs are lethal to cells as a result of the formation of DNA-top1-CPT ternary complexes. Relatively little is known about the interaction between top1 and other proteins, even though these interactions are likely important in the cellular functions of top1 and in the cytotoxic mechanisms of camptothecins. (Haluska et al., Adv. Enz. Regul., 1998). Interestingly, physical interactions were detected between top1 and two proteins implicated in carcinogenesis, SV40 T antigen and p53. (Haluska et al., Nucleic Acid Res., 1998; Zhou et al., 1999). These findings highlight the potential significance of top1-binding proteins in tumorigenesis.

Using a yeast two-hybrid screen, a novel topoisomerase 1- and p53-binding protein called topors was discovered. Topors is a RING protein that binds to the N-terminus of human top1. (Haluska et al., 1999). The coding region of topors is deposited under GeneBank Accession Number AF098300. Topors is unique in that it contains both a RING finger and serine and arginine domains in the same polypeptide. Subsequently, a peptide consisting of a fragment of topors as a p53-binding protein was identified. (Zhou et al., 1999). Homology searches indicate that the topors RING domain is similar to the RING domain of proteins involved in ubiquitin or SUMO transfer reactions.

Ubiquitination is critical to cellular function. The conjugated ubiquitin system tags proteins for degradation by proteosomes. As shown in FIG. 1, the ubiquitin activating enzyme activates ubiquitin in the presence of ATP. This enzyme is a single enzyme in most species. The ubiquitin conjugating enzyme contributes to substrate specificity. Ubiquitin ligase also confers substrate specificity and can be a complex of proteins (SCF) or a single protein (c-Cbl). Ubiquitin ligase may transfer ubiquitin directly from the ubiquitin conjugating enzyme to the substrate or form a ubiquitin-conjugate intermediate (See FIG. 1).

SUMO (small ubiquitin-related modifier) proteins are small protein tags that are conjugated to cellular regulator proteins by a set of enzyme proteins to modify their function. The regulator proteins include oncogenes and tumor suppressor genes that play key roles in the control of cell growth, differentiation and apoptosis. SUMO conjugation affect substrates' subcellular localization and stability as well as transcriptional activities. Three different SUMO proteins are conjugated to proteins, SUMO-1, SUMO-2 and SUMO-3. SUMO-1 is conjugated to proteins as a monomer, and SUMO-2 and SUMO-3 are conjugated to proteins as higher molecular weight polymers with SUMO-1 terminating further SUMO addition.

One target of SUMO modification is proteins involved in formation of the promyclocytic leukemia (PML) nuclear bodies. Acute promyelocytic leukemia, a type of cancer affecting the blood, is characterized by an abnormal block in the development of stem cells. Topors promotes the stability of PML nuclear bodies and perhaps alters their role in transcriptional regulation, cellular proliferation and anti-viral responses. The activity of several transcription factors is altered by sumoylation, including C/EBP proteins, c-Myb, glucocorticoid receptor, androgen receptor, and progesterone receptor. Sumoylation of topoisomerase I alters its localization in the nucleus, and histone deacetylase enzymes are targets of this system. Viral proteins are targets of sumoylation, suggesting that infection and anti-viral cellular defenses may be affected by this system. Protein sumoylation is important in cell cycle progression and genomic stability. Sumoylation may also alter the stability of proteins with polyglutamine repeats involved in neurodegenerative disorders, adding further to the important and diverse roles of this protein modification system.

Since Top 1 is appears to be an important potential anti-cancer drug target, it is clinically relevant to understand the function and expression of proteins, such as topors, that interact with Top 1. It is further important to understand the impact of a topors antibody. The present invention relates to the characterization of topors antibody, further characterization of topors, and the methods of using both topors and topors antibody for cancer diagnostics and therapeutics.

### SUMMARY OF THE INVENTION

Topors and topors antibody can be used to manipulate the presence and function of both Top1 and p53 in cells, thus controlling the function of the Top1 and p53 proteins. Topors is implicated in prevention of tumorigenesis through its role in DNA repair and preventing faulty or mutated DNA from replicating. Topors may also have a role in apoptosis of the defective cells.

The present invention contemplates the therapeutic use of the protein, creation and use of the antibody, the use of topors antibody in a kit for detection of cancer, or a kit to screen for the chance of tumorigenesis in the future. Various aspects of the invention are also directed toward using topors in uncontrolled proliferating cells, such as tumors, to increase sensitivity to cancer therapies and adding topors to inhibit or destroy tumor replicative function.

Further, the invention is directed to the use of a gene therapy method directed to topors production, either in vitro and in a subject. The topors may be used prophylactically to prevent cells from reaching functionally insufficient levels of topors. The gene therapy will also be directed to stimulating production of topors in uncontrolled proliferating cells, thus allowing the cells to either repair themselves or undergo apoptosis. These and other aspects of the present invention will be readily ascertainable when understood in conjunction with the following description and figures.

### BRIEF DESCRIPTION OF THE FIGURES

FIG. 1: Cartoon depiction of protein ubiquitination and sumoylation activities. E1 is the ubiquitin activating enzyme. E2 is the ubiquitin conjugating enzyme. E3 is ubiquitin ligase.

FIG. 2: A polyclonal topors antibody recognizes both recombinant and endogenous topors. 50 μg of HeLa cell lysates obtained from cells transiently transfected with a GFP-topors plasmid and 100 μg of colon tissue lysate were loaded in lanes 1 and 2 in each panel, respectively. Immunoblotting was performed with the topors polyclonal antibody. Migration of GFP-topors and endogenous topors are indicated.

FIG. 3: Topors protein levels in normal and tumor endometrial tissues. Each lane contains 10 μg of protein. Immunoblotting analyses were performed using a topors polyclonal antibody. The blots were stripped and reprobed with actin monoclonal antibody.

FIG. 4: Topors protein levels in normal and tumor tissues from colon, kidney and lung. 10 μg of proteins were loaded in each lane and immunoblotted with topors polyclonal antibody. The blots were then analyzed with actin monoclonal antibody.

FIG. 5: Topors mRNA expression in normal and tumor tissues. Total RNA was isolated from tissue samples and analyzed simultaneously for topors and G3PDH mRNA expression using specific primers and RT-PCR.

FIG. 6: Topors enhances top1 sumoylation. Purified top1 was incubated with GST-topors, SAE1/SAE2, Ubc9, and SUMO-1 for 2 hours at 30° C. The sumoylation reaction was stopped by adding sample buffer and heating for 5 min at 95° C. The samples were resolved by SDS-PAGE and transferred to nitrocellulose. Western blotting was carried out using top1 and SUMO antibodies.

FIG. 7: Gels showing that topors functions as an E3 ubiquitin ligase in vitro. Ubiquitin was incubated with GST, topors, and E2. The location of the poly-ubiquitin chains and the free ubiquitin is shown to the right of the gels.

FIG. 8: Gels indicating that topors functions as an E3 SUMO ligase for Top 1. The location of free top1 and top1-sumo conjugates is shown to the left of the gels.

FIG. 9: Topors also activates sumoylation of p53, but not IκBα, showing that proteins subject to sumoylation are not indiscriminately activated by topors.

FIG. 10: Topors increases SUMO-2 conjugates in mammalian cells. The presence or absence of GFP, Flag-SUMO-2, and GFP-topors is indicated in both H1299 and HeLa cells for various samples.

FIG. 11: Topors associates with PML nuclear bodies. The top row of images shows GFP-topors, PML, and an overlay of both images. The bottom row shows endogenous topors, PML, and an overlay of both images.

FIG. 12: Topors relocalizes to the nucleoplasm after DNA damage. Topors protein is labeled and imaged at time 0 and at 30 minutes.

FIG. 13: Image of normal and tumor samples subjected to SDS-PAGE and immunoblotting show a loss of topors protein expression in colon cancers.

FIG. 14: Agarose gel showing a differential expression of topors mRNA in matched normal and colon cancer. The numbers at the top of each panel refer to a matched number specimen.

FIG. 15: A cartoon depiction of a model for the cellular function of topors. PML NB stands for promyelocytic leukemia nuclear bodies. CPT stands for camptothecin.

FIG. 16: An agarose gel showing topors (lower panel) mRNA and β-actin (upper panel). Topors mRNA is expressed in most normal tissues.

FIG. 17: A bar graph showing that overexpression of topors inhibits DNA synthesis in HeLa cells.

### DETAILED DESCRIPTION OF THE INVENTION

Definitions

The present invention may be best understood in conjunction with these definitions:

“Antibodies” as used herein includes polyclonal and monoclonal antibodies, chimeric, single chain, and humanized antibodies, as well as Fab fragments, including the products of a Fab or other immunoglobulin expression library.

“Monoclonal antibodies” means substantially homogenous populations of antibodies to a particular antigen. They may be obtained by any technique that provides for the production of antibody molecules by continuous cell lines in culture. Monoclonal antibodies may be obtained by methods known in the art.

“Polyclonal antibodies” are a group of heterogeneous antibodies produced by different B lymphocytes in response to the same antigen, wherein different antibodies in the group recognize different parts of the antigen.

The term “specific binding affinity” means that the antibody or antibody fragment binds to target compounds (i.e., topors) with greater affinity than it binds to other compounds under specified conditions. Antibodies or antibody fragments having specific binding affinity to a compound may be used to inhibit the function of that compound by contacting it with the antibody or antibody fragment under conditions such that an immunocomplex forms, inhibiting the function of the compound conjugated to the antibody or antibody fragment. Alternatively, the antibody may be used to bind the topors protein and identify topors' presence in a given sample.

A “pharmaceutically acceptable carrier” is one that is non-toxic to recipients at the dosages and concentrations employed and is compatible with other ingredients of the formulation. For example, the formulation would not contain any substances that are known to be deleterious to topors. The carrier may contain additives such as substances that enhance isotonicity and chemical stability. The additive materials may include buffers such as phosphate, citrate, succinate, acetic acid, and other organic acids or their salts; antioxidants such as ascorbic acid; low molecular weight (less than about fifteen residues) polypeptides, proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, trehalose, glucose, mannose, or dextrins; chelating agents such as EDTA; sugar alcohols such as mannitol or sorbitol; counter-ions such as sodium; and/or nonionid surfactants such as polysorbates, poloxamers, or PEG. The final topors preparation may be a liquid or lyophilized solid. Topors, a suitable derivative or metabolite thereof, may be used alone or in admixture with one or more additional active agents.

“Therapeutically effective amount” refers to that amount of the compound that results in amelioration of symptoms or a prolongation of survival of a subject. A therapeutically effective amount of topors in the present invention will generally be in the range of about 0.01 μg/kg to about 100 mg/kg per day. Preferably, from 0.1 to 1 μg/kg. A clinician will administer topors formulations of the invention until a dosage is reached that improves uncontrolled proliferating cells condition, taking into account the usual factors the specific disorder being treated and the severity of the disorder, the specific composition administered, the age, weight, general health, and gender of the subject, and other factors individual to the subject. The progress of this therapy can be monitored by usual assays for detecting the disorder. For example, if the disorder is cancer, the progress of cancer treatment can be monitored through blood test, CAT scans, PET scans, urinalysis, and other known methods.

The term “therapeutically effective amount” with respect to a vector refers to a dose of vector and level of gene expression resulting from the action of the promoter on the nucleic acid cassette when introduced into the appropriate cell type that will produce sufficient protein, polypeptide, or antisense RNA to either increase the level of protein production, decrease or stop the production of a protein, inhibit the action of a protein, inhibit proliferation or accumulation of specific cell types, or induce proliferation or accumulation of specific cell types. The dose will depend on the protein being expressed, the promoter, uptake and action of the protein or RNA.

“Administration” means administration of the therapeutic compound. Administration may be carried out orally, parenterally, osmotically, or topically in unit dosage formulations containing carriers, adjuvants, diluents, vehicles, or combinations thereof. “Parenteral” includes infusion as well as subcutaneous, intravenous, intramuscular, and intrasternal injection. Parenterally administered aqueous compounds may be formulated with dispersing, wetting, or suspending agents. Use of diluents or solvents are also acceptable if they do not significantly alter the pharmaceutical effectiveness of the topors being administered. Among the acceptable diluents or solvents employed are water, saline, Ringer's solution, buffers, monoglycerides, diglycerides, fatty acids such as oleic acid, and fixed oils such as monoglycerides or diglycerides. Materials may be used to slow the absorption of parenterally administered compounds, such as suspensions of crystalline, amorphous, or otherwise water-insoluble forms of the compound.

Oral administration of solid dosages include capsules, tablets, pills, powders, and granules. Here again, diluents and buffering agents such as sucrose, lactose, starch, talc, silicic acid, aluminum hydroxide, calcium silicates, polyamide powder, tableting lubricants, and tableting aids are acceptable. Excipients like talc, silicic acid, aluminum hydroxide, calcium silicate, polyamide powder, may be added to powders and sprays. Potential forms of liquid dosage include emulsions, microemulsions, solutions, suspensions, syrups, and elixirs. Topical administration may occurs through the use of ointments, pastes, creams, lotions, gels, powders, solutions, sprays, inhalants, and/or transdermal patches, all of which may also comprise excipients. For administration by inhalation, the compounds for use according to the present invention may be delivered in the form of an aerosol spray presentation from pressurized packs or a nebuliser, with the use of a suitable propellant, like dichlorodifluoromethane, trichlorofluoromethane, dichlorotetrafluoroethane, carbon dioxide or other suitable gas.

Finally, administration may be local or systemic, depending on the location of the cells or tissues to be treated. For example, the drug may be administered in a targeted drug delivery system, such as in a liposome coated with a specific antibody, targeting the affected tissue. The liposomes will be targeted to and taken up selectively by the afflicted tissue. It is also contemplated to administer the pharmaceutical composition locally with an implant or device.

By “nucleic acid” is meant both RNA and DNA including cDNA, genomic DNA, plasmid, DNA, condensed nucleic acid, or nucleic acid formulated with compounds able to prolong the localized bioavailability of a nucleic acid. In one preferred embodiment, the nucleic acid administered is plasmid DNA that comprises a “vector”.

The term “vector” refers to a construction comprised of genetic material designed to direct transformation of a targeted cell, as well as, various regulatory elements for transcription, translation, transcript stability, replication, and other functions as are known in the art. “Post-translational processing” means alterations made to the expressed gene product, including addition of side chains such as carbohydrates, lipids, inorganic or organic compounds, and cleavage of targeting signals or propeptide elements. The vector may comprise one or more genes in a linear or circularized configuration, or a plasmid backbone.

An “expression vector” is a vector that allows for production of a product encoded for by a nucleic acid sequence contained in the vector. For example, expression of a particular growth factor protein encoded by a particular gene. A “viral vector” is a vector whose orginal form is as the genomic material of a viral particle. Viral vectors include retrovirus, adenovirus, adeno-associated virus, and lentivirus.

The term “promoter” refers generally to transcriptional regulatory regions of a gene, which may be found at the 5′ or 3′ side of the coding region, or within the coding region, or within introns. A promoter is usually DNA regulatory region capable of binding RNA polymerase in a cell and initiating transcription of a downstream coding sequence. The typical 5′ promoter sequence is bounded at its 3′ terminus by the transcription initiation site and extends upstream to include the minimum number of bases or elements necessary to initiate transcription at levels detectable above background. Within the promoter sequence is A transcription initiation site is found within the promoter sequence.

“Plasmid” means a vehicle comprised of extrachromosomal genetic material, usually of a circular duplex of DNA that can replicate independently of chromosomal DNA. Plasmids may be used in gene transfer as vectors.

The term “ligand,” as used herein, refers to any compound or molecule that binds to and activates a receptor.

“Mutated” refers to an alteration of the primary sequence of a receptor or any other gene or protein such that it differs from the wild type or naturally occurring sequence.

The term “expression cassette” refers to the combination of nucleic acid sequences involved in expression of a particular functional product, which may be any form of nucleic acid. The expression cassette may also be comprised of non-coding regions in addition to sequences encoding a product such as a protein.

Embodiments

The first preferred embodiment of the present invention is the creation of the topors antibody itself. The antibody may be either polyclonal or monoclonal. Preferred are antibodies that effectively bind to topors and completely inhibit topors activity with respect to topors binding to Top 1, p53, or any other protein with which topors interacts. Most preferred are topors antibodies with specific binding affinity for the topors protein alone. The topors antibody and gene sequence will also be useful for research involving the further investigation of cellular role of topors and related nuclear proteins.

Monoclonal antibodies may be prepared by standard according to general hybridoma methods of Kohler and Milstein, Nature (1975) 256:495-497), the trioma technique, the human B-cell hybridoma technique (Kozbor et al., Immunology Today (1983) 4:72) and the EBV-hybridoma technique (Cole et al., Monoclonal Antibodies And Cancer Therapy, pp. 77-96, Alan R. Liss, Inc., 1985). Antibodies utilized in the present invention may also be polyclonal antibodies, although monoclonal antibodies are preferred because they may be reproduced by cell culture or recombinantly, and may be modified to reduce their antigenicity. Polyclonal antibodies may be raised by a standard protocol by injecting a production animal with an antigenic composition, formulated as described above. See, e.g., Harlow and Lane, Antibodies: A Laboratory Manual, Cold Spring Harbor Laboratory, 1988.

In a second preferred embodiment, a topors antibody may be used to detect the presence or absence of topors in various screens, which may lead to improved diagnosis and treatment of cancer and other topors related diseases. The screen will preferably indicate the absence or presence of topors in cancer and/or normal tissues by removing material to be tested from a subject, applying the topors antibody, and measuring the specific binding of the antibody. Preferably, the antibody is detectably labeled. Most preferably, the antibody is detectably labeled and easily assayed, such as with fluorescence. If the topors antibody binds to topors, then topors is present in the sample, indicating that cancer is not present.

In a highly preferred embodiment, the amount of topors per sample, and not just its presence or absence is detected. Detection of the relative and/or absolute amount of topors will allow diagnosticians to determine if the levels of topors are normal or depressed. Depressed levels of topors could indicate that uncontrolled cellular proliferation does not currently exist, but has a higher chance of occurring in that subject in the future. This use of the topors antibody leads into the next preferred embodiment, which is using the antibody screen to predict a subject's chances of contracting an uncontrolled proliferating cellular disease, such as cancer. A topors antibody screen could allow subjects to test various tissues to determine whether the presence of topors was diminished. If diminished levels of topors protein were found, a treating physician could take proactive steps to prevent the cancer, such as administering topors to the subjects via the embodiments described below.

In a related embodiment, a genetic screen can be employed to identify subjects with higher susceptibility to tumorigenesis based on loss of normal topors DNA or RNA before cancer or any other disease related to the loss of topors manifests in a subject. Subjects with mutated DNA or RNA encoding topors would have a greater chance of those cells not producing topors or producing a nonfunctional form of topors. The genetic screen could be carried out by methods well known in the art, such as isolating a gene using restriction enzymes and then sequencing the gene or examining the gene for polymorphisms.

Other aspects of the invention also uses the topors antibody as a screen in a similar manner, examining samples for higher than normal (rather than lower) physiological levels of topors. The screen would be used to detect a disease in which topors is overexpressed in tissues, such as diseases wherein cells are subject to high levels of cellular stress. Recent data have revealed the relevance of posttranslational modification of proteins via covalent attachment of SUMO in cell cycle progression, stress response and signal transduction. p53 functions in response to a variety of cellular stresses. While the beneficial anticancer effects of p53 are well established, p53 has also been implicated in human aging. (Sharpless et al., 2002).

If the screen determined that the cellular level of topors were elevated, another aspect of the invention would instruct the administration of topors antibody in vivo to the location of the elevated topors levels. The topors antibody would bind topors. Thus, by controlling the level of topors, which binds to p53, cellular aging could be manipulated, particularly if it were previously exposed to a stress that raised the level of p53. An important aspect of this embodiment is to maintain monitoring of and control over the level of topors in the cells because depressed levels of topors lead to uncontrolled cellular proliferation.

A kit for prediction and diagnosis of diseases related to uncontrolled cellular proliferation, such as cancer, by using the topors antibody for all of the methods described above is also contemplated. A kit for this purpose would comprise the topors antibody and instructions for use. Since the antibody can be used in multiple ways to carry out the multiple methods described, the directions could indicate each of the ways in which the screening method can be used. The kit could also contain reagents, a detectable label for the antibody, tubes, trays, and other items associated with the carrying out of the screen.

In yet another embodiment, the present invention teaches a method of using topors as a tumor suppressor protein. Both topors and the topors antibody can be used to modulate DNA repair process. A therapeutically effective amount of topors could be administered in a pharmaceutically acceptable carrier if a cell with mutated DNA were replicating in order to stop the continued production of the DNA. If a normal and/or advantageous cell had lost the ability to replicate due to an over-abundance of topors, topors antibody could be added to bind up some of the excess topors to allow the cell to continue replicating. The topors could be administered generally through a variety of routes of administration, or locally to the site of uncontrolled cellular proliferation.

An additional aspect of the present invention contemplates the addition of topors to uncontrolled proliferating cells, not just for the correction of the nucleic acid or apoptosis by allowing the camptothecins to work effectively, but also to sensitize the cancerous cells to traditional cancer therapy treatments. The topors makes the cells ready to either repair DNA damage or die. The addition of chemotherapy or radiation further weakens the cells and likely causes those cells to self-destruct.

Yet another embodiment of the present invention involves the use of the topors gene sequence and protein to develop small molecule inhibitors for topors ubiquitin and SUMO ligase activities. Both the ubiquitination and sumoylation activities assist in controlling the replication of the cells and the small molecule inhibitors would allow for further manipulation of these processes. The small molecule inhibitors would be developed through methods known in the art given the knowledge of the sequence and function of topors as a tumor inhibitor.

Finally, the topors sequence could also be used in a gene therapy approach to replace or re-introduce topors DNA as the therapeutic gene into the desired location in a subject or in vitro. Preferably, the area of delivery would be a tumor cells and topors sequence would be enclosed in a construct, such as a vector. The vector could be a viral vector, or a nonviral vector, such as a liposome. Post translational processing of the DNA may occur after the DNA has been translated by the affected cell. In a further embodiment, topors antibody could be used in a vaccine to treat diseases associated unchecked cell proliferation. The gene therapy system may be inducible, meaning that the genes are only transcribed when an outside stimulus is applied. Promoters may also be used in the vector.

Overview of Findings

The data indicate that topors regulates top1 by functioning as an E3-type SUMO ligase for top 1. It has been shown that following exposure to CPT, top1 is sumoylated and ubiquitinated, followed by downregulation in normal cells, but not in tumor cells (Rasheed, 2001). Thus, topors is a tumor suppressor.

It is likely that the relative lack of topors in tumor cells may be involved in this differential response to CPT. In normal cells, DNA damage induces an interaction between topors and top1, resulting in the inactivation of top1 via sumoylation, which facilitates the DNA repair process. However, in cells lacking topors, top1 activity remains unchanged in the presence of DNA damage. This persistence of top1 activity may enhance DNA mutagenesis, a phenotype that is selected for in carcinogenesis. Furthermore, topors protein was detected in normal human tissue samples but not in matched human tumor tissue specimens from kidney, colon, endometrium and lung using the topors-specific antibody of the present invention. In the tissue specimens where topors protein levels were undetectable in tumor, little if any, topors mRNA was detected. For example, endometrium and colon tumor tissue samples lacking topors protein did not reveal measurable mRNA levels. This finding is consistent with the protein data.

Topors functions as an E3-type SUMO ligase for Top1 and p53 in a purified in vitro system. Furthermore, using a polyclonal antibody developed against recombinant full-length topors, it is demonstrated that the topors protein is expressed in several normal tissues, including bladder, colon, endometrium, kidney, lung, and prostate. Similarly, analysis of a panel of normal tissue cDNAs indicates that topors mRNA is detectable in most normal tissues. The experiments also analyzed 8 matched tumor/normal tissue specimens obtained from patients with colon (4 patients), endometrial (1 patient), lung (1 patient), or renal (2 patients) cancers for actin and topors protein expression. Although actin levels were similar in the tumor and normal tissues, topors protein levels in tumor tissues were either undetectable or significantly less than levels found in corresponding matched normal tissues, for all but one colon tissue pair. Similar results were obtained with quantitative multiplex RT-PCR studies, which indicated loss of topors mRNA expression in 2 colon tumors and one endometrial tumor, relative to matched normal tissue obtained from the same patients. The topors gene maps to chromosome 9p 13 in a region that exhibits loss of heterozygosity or homozygous deletion in several different tumor types. Together, these results suggest that loss of topors SUMO ligase activity for Top1, p53 , or other substrates may predispose to malignancy. Given the substrates involved, protein sumoylation is important in the course of tumorigenesis and, accordingly, altered in human cancer.

Furthermore, overexpression of Topors in cervical cancer cell lines leads to cell death. Thus, a lack of topors in cancer cells contributes to the selection and persistence of mutant phenotype and progression to tumorigenesis. Thus, topors can be added directed to tumor cells to induce cell death. Topors could also be added to tumors to make the tumor cells more sensitive to anti-cancer therapies, such as radiation and chemotherapy.

Additionally, it was shown that topors functions as an E3-type ubiquitin ligase an E3-type SUMO ligase for topoisomerase and p53. Thus, topors is a dual function ubiquitin and SUMO ligase. Recombinant topors enhances formation of polyubiquitin conjugates by specific E2 ubiquitin enzymes in vitro, with the RING domain necessary and sufficient for this activity. While topors-induced ubiquitination of bacterially expressed top1 was unable to be detected in vitro, similar assays using Ubc9, SAE1/SAE2, and SUMO-1 indicate that topors stimulates formation of top1 -SUMO-1 conjugates by Ubc9. Additional in vitro studies indicate that topors enhances Ubc9-mediated sumoylation of p53 but not IκBα, suggesting that topors does not non-specifically increase Ubc9 activity. Stimulation of top1 sumoylation by topors does not require the topors RING domain. Instead, this activity maps to the 536-704 region, which contains a nuclear localization sequence and is within the top1-binding region. Ectopic expression of topors in HeLa cells increases formation of SUMO-2 conjugates.

Collectively these data indicate that Topors is a candidate tumor suppressor gene similar to p53 and the loss of topors SUMO ligase activity could lead to cancer. Furthermore, it is possible that modulation of topors ubiquitin and/or SUMO ligase activities may be useful in diseases associated with alterations in ubiquitin or SUMO pathways, including cancer.

In summary, topors protein is expressed in most normal tissues and functions as a dual E3 ubiquitin/SUMO ligase in vitro and is the first known protein with this activity. Topors expression is lost in colon and other common human cancers, however, indicating that topors functions in the cellular response to DNA damage.

### EXAMPLES

The following examples are intended to illustrate the invention, not limit it.

### Example 1:

### Generation of Anti-Topors Polyclonal Antibodies

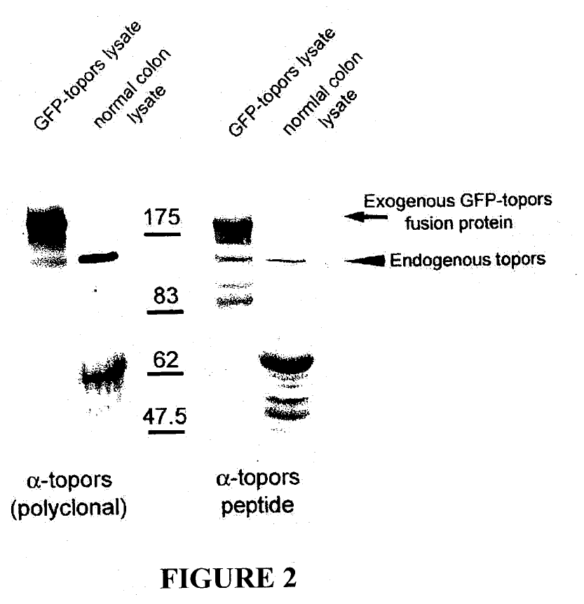

Topors cDNA was cloned into pKG, an inducible yeast expression vector, to obtain purified GST-topors from a eukaryotic source. The purified recombinant GST-topors protein was used to immunize rabbits. Similarly, a peptide representing residues 870-889 (VYEGKATDTTKHHKKKKKKH) [SEQ ID NO: 1] of topors was used to generate antibodies directed towards this region of the protein. Immunoblotting analyses indicate that both antibodies recognize a recombinant GFP-topors protein in HeLa cell lysates (FIG. 2). Furthermore, both antibodies recognize a predominant band migrating at ˜135 Mᵣ in normal bladder, colon, prostate, endometrium, kidney, lung, stomach and testicles tissue lysates (FIG. 2). This band is also recognized, albeit with lower affinity, by an antibody recognizing a C-terminal peptide of topors. Neither the GFP-topors or endogenous topors bands were visualized in control experiments using pre-immune rabbit serum or secondary antibody alone (data not shown).

As expected, the serum from rabbits injected with the full-length GST-topors protein yielded a better signal in immunoblotting assays (FIG. 2), and this serum was used in all subsequent topors immunoblotting experiments. In eukaryotic cells topors migrates slower than predicted based upon calculated molecular weight (predicted molecular weights of GFP-topors and endogenous topors are 146 and 119 kDa, respectively, with these proteins migrating at 170 and 135 Mᵣ, respectively). This aberrant migration may be due to conjugation with SUMO proteins.

### Example 2:

### Differential Expression of Topors protein in Matched Tumor and Normal Tissues

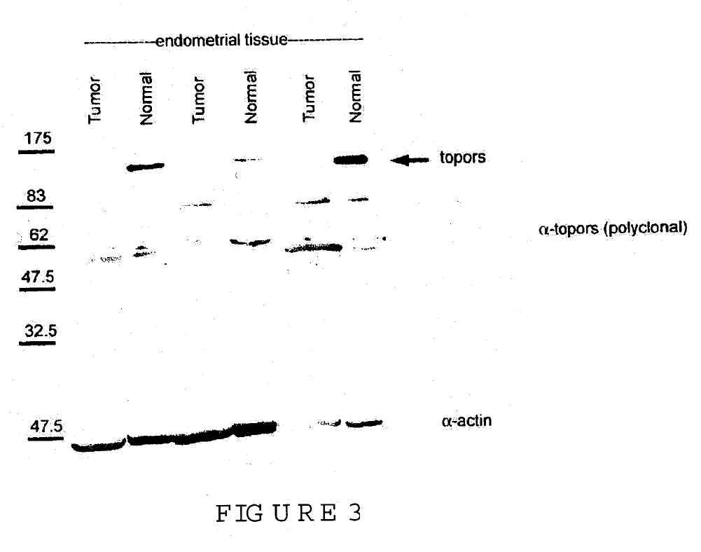

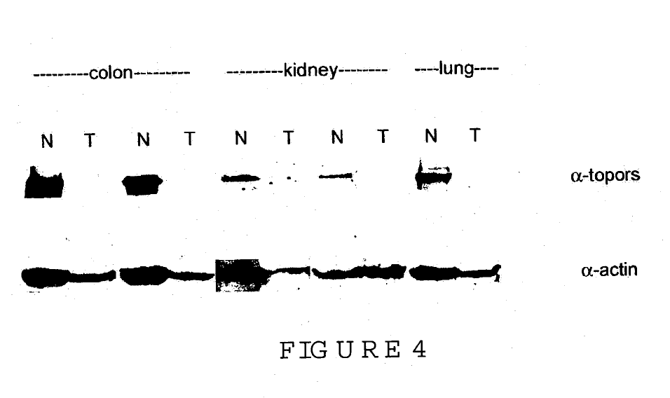

The topors protein was not detected in lysates from several different tumor cell lines (data not shown). However, in matched normal and tumor human tissue specimens, the topors protein was detectable only in normal tissues (FIGS. 3 and 4). As a control, the same blots were reprobed with an actin monoclonal antibody, with results indicating that actin levels were similar, excluding unequal loading as a reason for the difference in topors levels between normal and tumor tissues. In addition, overexpression of topors in a cervical cancer cell line leads to cell death. It is possible that expression of topors in cancer cells leads to alterations in the sumoylation of top 1 and other proteins that are required for uncontrolled proliferation, resulting in tumor cell death.

### Example 3:

### Differential Expression of Topors mRNA in Tumor versus Normal Tissues

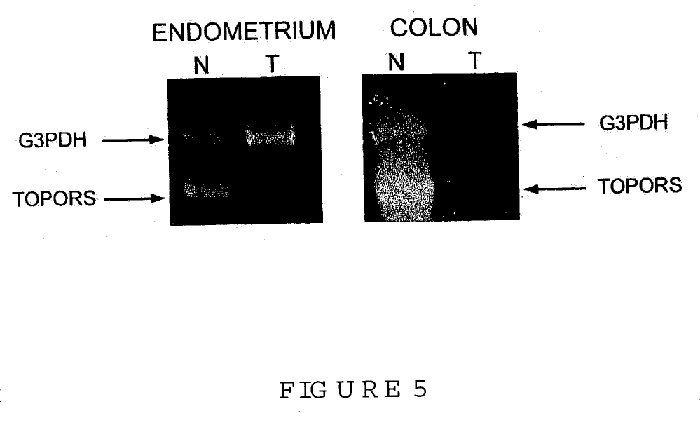

In two specimens where topors protein levels were undetectable in tumor, little, if any, topors mRNA was detected by RT-PCR, whereas G3PDH (glyceraldehyde-3-phosphate dehydrogenase) mRNA bands were similar in tumor and normal tissues (FIG. 5). The significance of this finding is that not all mRNA was lacking in tumor cells-non-topors mRNA remained at normal physiological levels.

### Example 4:

Topors function as an E3-type SUMO ligase, catalyzing SUMO conjugation to Top I

Topors is homologous in the RING domain to proteins implicated in conjugation of ubiquitin and SUMO, such as MDM2 and BRCA1. Previously, we showed that topors binds to the N-terminus of top1. Furthermore, top1 has been shown to be a substrate for ubiquitin- and SUMO-conjugation following cellular treatment with CPT. Topors also redistributes from PML (promyelocytic leukemia) nuclear bodies to a diffuse nuclear localization when exposed to CPT. When incubated with a HeLa cell fraction containing SAE1/SAE2, purified SUMO-1, and purified Ubc9, minimal top1-SUMO conjugates can be detected using Top1 and SUMO antibodies.

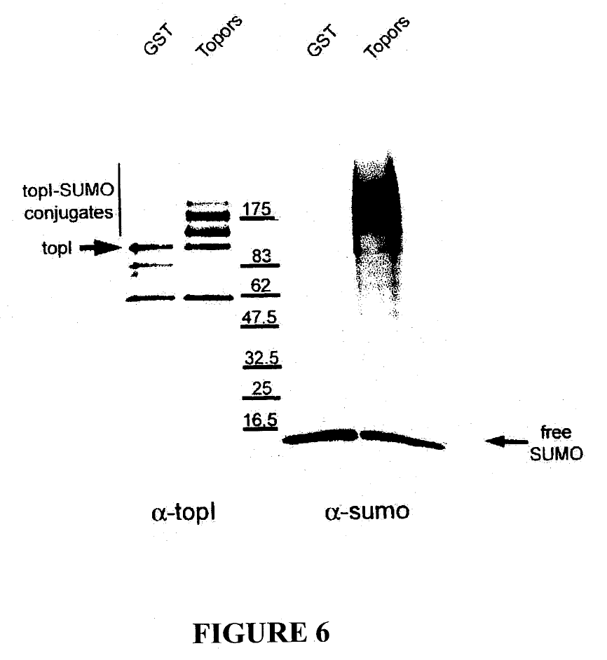

The present experiments show that GST (Glutathione S-Transferase)-topors binds to the SUMO-conjugating enzyme (Ubc9), whereas GST does not bind. Furthermore, they also show that topors enhances top1 sumoylation in vitro (FIG. 6). Using purified components of the sumo conjugation system as well as purified top1 and topors, top1-SUMO conjugates were detected. Removing any of the sumoylation components or topors abrogated top1 sumoylation. Taken together, these data show that topors functions as an E3-type SUMO ligase, catalyzing top1 sumoylation.

### Example 5:

### Topors Functionality and Cellular Behavior

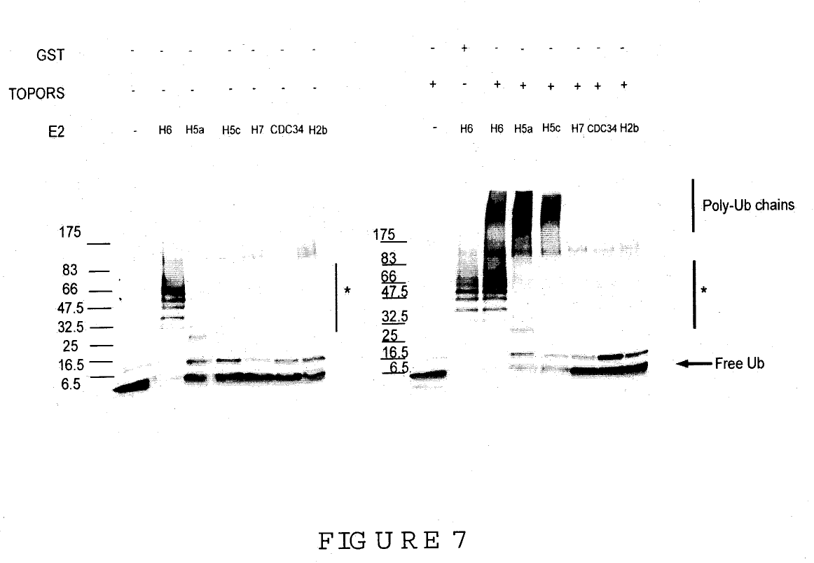

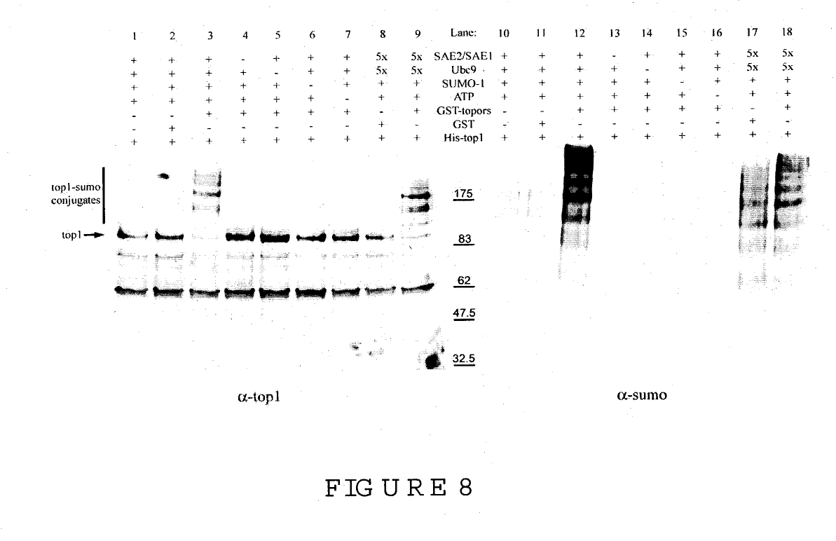

Topors functions as an E3 ubiquitin ligase (FIG. 7) and as an E3-type SUMO ligase for Top 1 (FIG. 8), both of which act to regulate the replication of DNA in a given cell. In FIG. 7, reactions contained 400 ng E1, 200 ng of the indicated E2, 3 μg of ubiquitin, and as indicated either˜100 ng GST-topors or GST. Reaction products were resolved by SDS-PAGE under reducing conditions. Migration of probable poly-ubiquitin chains is indicated. The asterisk indicates migration of ubiquitin conjugates observed in the presence of the E2 alone. All E2s are active as assessed by thiolester formation (not shown). In FIG. 8, the reactions contained 100 ng or 500 ng (5×) SAE2/SAE1, 30 ng or 150 ng (5×) UbcH9, 200 ng SUMO-1, 4 mM ATP, 100 ng GST-topors or GST, and 250 ng His-top1. His-top1 is indicated by an arrow.

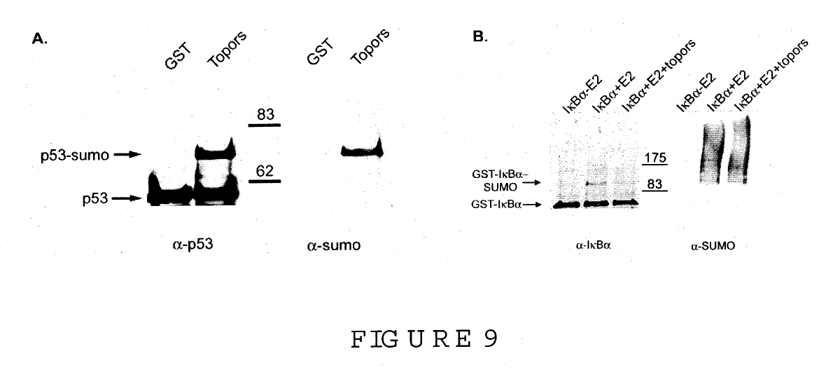

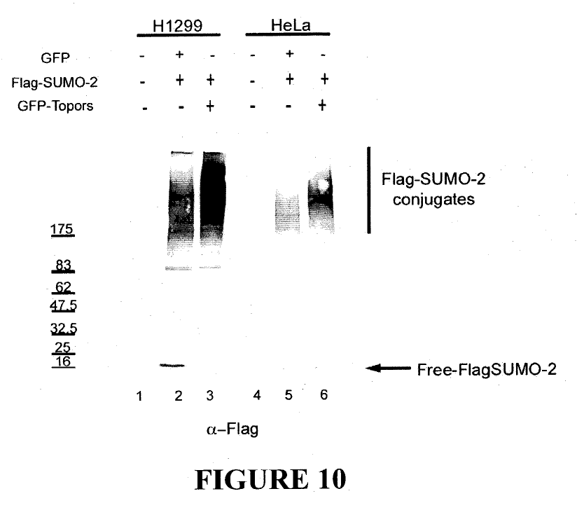

Topors also activates sumoylation of p53, but not IκBα. This finding is significant because is known to be sumoylated and IκBα is a check to ensure that topors does not activate p53 and Ubc9 nonspecifically (FIG. 9). In FIG. 9, reaction products were analyzed using monoclonal anti-p53 (A) and polyclonal anti-IκBα (B) antibodies. Reactions in B contain increased amounts of E1 and Ubc9 relative to reactions in A, in order to demonstrate sumoylation of IκBα by Ubc9 in the absence of topors. In addition, topors increases SUMO-2 conjugates in mammalian cells (FIG. 10).

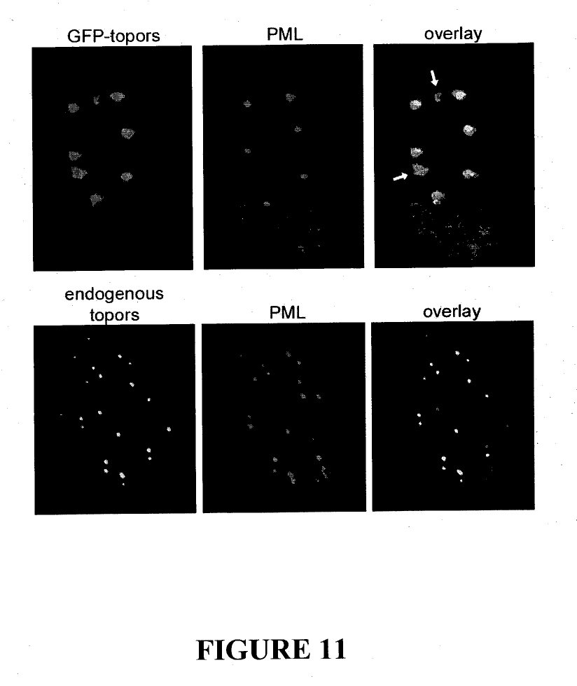

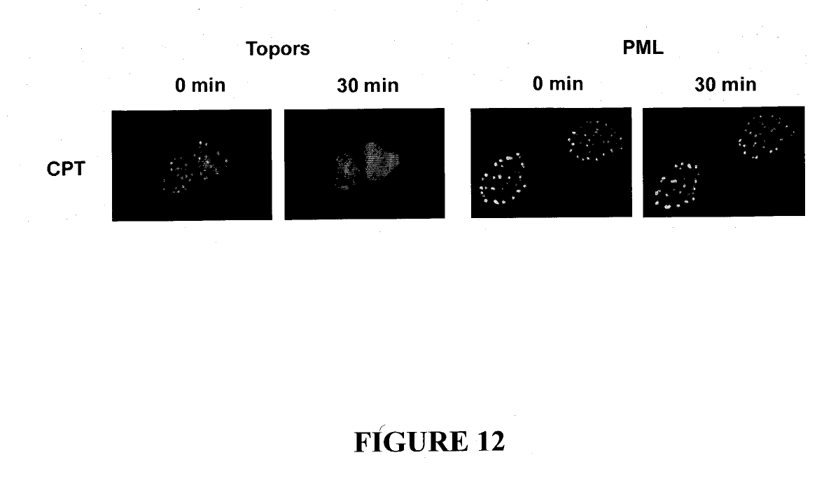

Acute promyelocytic leukemia, a type of cancer affecting the blood, is characterized by an abnormal block in the development of stem cells. It has also been discovered that topors localizes in nuclear bodies associated with promyelocytic leukemia (PML) oncogenic domains (FIG. 11). In FIG. 11, HeLa cells were immunostained with antibodies recognizing both topors and PML. Separate and merged fluorescent images from a representative cell are shown. PML nuclear bodies are nuclear depot sites, which are disrupted in cells from patients with promyelocytic leukemia with t(15; 17). Topors relocalizes to the nucleoplasm after DNA damage (FIG. 12). FIG. 12 shows that DRB and CPT induce rapid dispersion of topors but not PML from nuclear bodies. As indicated, HeLa cells expressing GFP-topors or GFP-PML were imaged before and after treatment with 0.1% DMSO, 10 μM DRB, 10 μM CPT, or 30 μM cycloheximide for thirty minutes.

### Example 6:

### Loss of topors protein and mRNA expression in human colon cancers relative to matched normal colon tissue

Using the topors antibody derived against the full-length protein, topors protein expression was surveyed in cancer cell lines and in human normal and cancer tissues (obtained from the CINJ Tissue Retrieval Core and the Cooperative Human Tissue Network). Gross diagnosis of the tissues was reconfirmed by H&E staining. After thawing, frozen tissue specimens were weighed and cut into approximately 0.1 g aliquots. For protein analysis, an aliquot was homogenized in 1 ml of buffer containing 50 mM Tris pH 7.2, 150 mM NaCl, 1 mM PMSF, 0.5 μg/ml leupeptin, and 1 μg/ml pepstatin. SDS was then added to 1%, the lysates incubated at 95° C. for 10 min., then centrifuged at 13,000×g for 10 min. SDS-PAGE sample buffer was added to the supernatant, which was heated at 95° C. for 5 min, then loaded onto a polyacrylamide gel. The samples were first analyzed for α-actin expression, then diluted in lysis buffer as needed to obtain relatively equal concentrations of this protein. α-actin-normalized samples were then subjected to SDS-PAGE and immunoblotting using the topors antibody.

In FIGS. 9A and 9B, results obtained with 2 sets of normal (N) and colon cancer (T) tissues are shown. In panel B, “H” represents ˜20 μg of lysate obtained from Hct116 colon cancer cells, and lanes with asterisks represent matched normal and cancer tissues obtained from the same patient. Note that in the single asterisk case, the tumor tissue was adenoma, whereas the tumor tissue was adenocarcinoma in all other cases. Ponceau staining of each blot is shown, as well as results obtained by immunoblotting with α-actin (A) or top1 (B) antibodies.

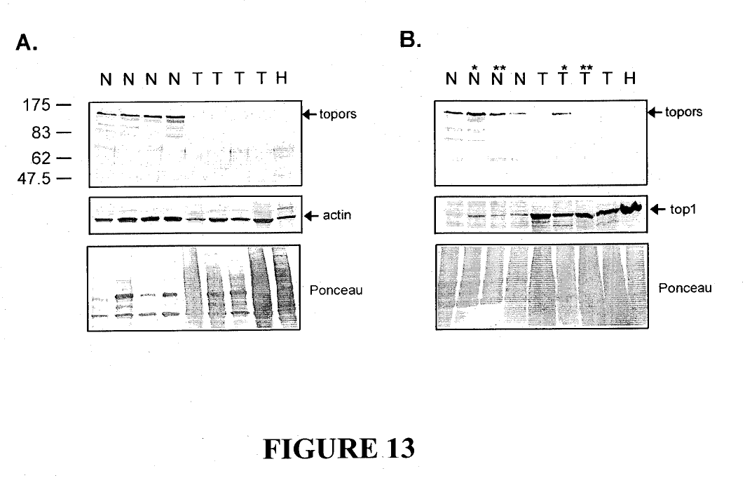

It was difficult to detect topors expression in several cancer cell lines (U-937, HeLa, Hct116, DU145, MCF7), but not in normal tissues, including bladder, colon, endometrium, kidney, lung, and prostate (FIG. 13B for Hct116, others not shown). To further investigate this phenomenon, a series of matched and unmatched specimens of normal colon tissue and colon tumors (8 adenocarcinoma, 1 adenoma) was analyzed for expression of the topors protein. When the samples were normalized for α-actin expression, topors protein expression was detectable in all the normal colon specimens, with expression levels greater in some specimens than in others (FIG. 13). Topors protein expression was also detectable in the benign adenoma specimen (FIG. 13,T*). By contrast, topors protein expression was either low or undetectable in several colon cancer specimens. Currently, 14 colon cancer specimens have been analyzed and topors protein expression was detected in only two of those specimens. Furthermore, the relative decrease in topors expression in cancer versus normal tissues is likely underestimated using the α-actin normalization approach, since Ponceau staining indicates that this approach typically results in significantly more protein being loaded in cancer tissue lanes (FIG. 13). In addition, these results cannot be explained by decreased extraction of nuclear proteins from cancer versus normal tissues, since top1 levels are typically higher in colon cancer versus normal colon tissues (FIG. 13B).

### Example 7:

### Differences in Expression between matched normal and cancer colon tissue of topors mRNA expression

Topors mRNA expression was also analyzed in a series of 10 matched colon tumor/normal specimens (9 adenocarcinoma, 1 adenoma), using a semi-quantitative multiplex RT-PCR assay. Topors primers consisted of a 5′-primer in exon 2 and a 3′-primer in exon 3 (yielding a 264 bp band). Glyceraldehyde-3-phosphate dehydrogenase (G3PDH) primers (yielding a 982 bp band) served as a control for sample RNA content. RNA extraction from matched normal colon (N) and colon cancer (T) tissues was performed using the PUREscript RNA Isolation Kit (Gentra Systems, Inc.). PCR products were visualized by agarose gel electrophoresis, followed by ethidium bromide staining. Note that in specimen 1545 the tumor is a benign adenoma rather than adenocarcinoma, which is the case for all other specimens.

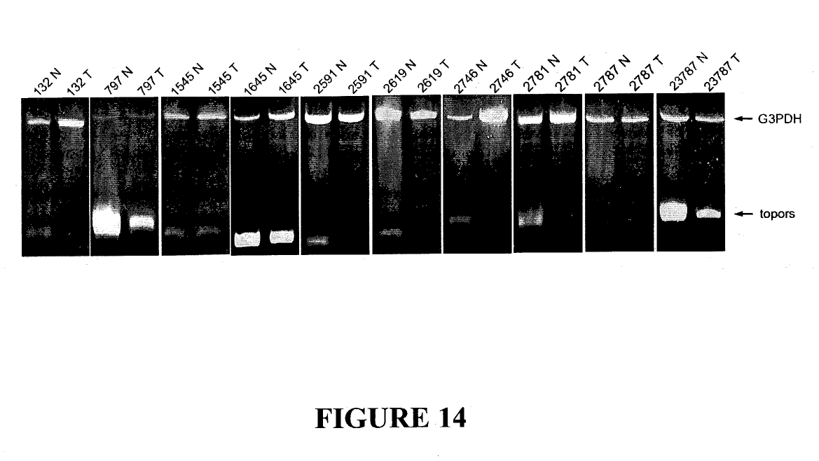

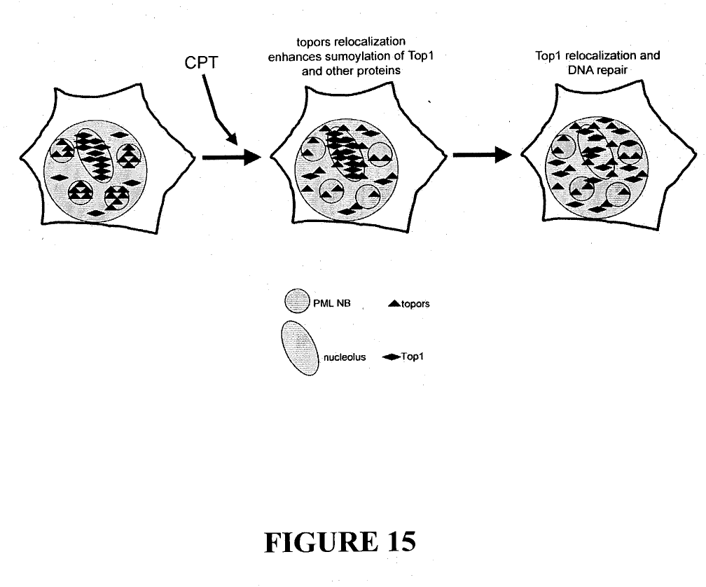

Similar to results obtained with the topors antibody, we found that topors mRNA expression was reduced in 7 of 10 colon cancer tissues relative to matched normal tissues (FIG. 14). Furthermore, topors mRNA expression was compared to topors protein expression for two matched specimens. Both topors protein and mRNA expression were similar in the normal and tumor (adenoma) tissue in one case (denoted as a single asterisk in FIG. 13B, and as 1545N and T in FIG. 14), whereas in the other case, both topors protein and mRNA expression were reduced in the cancer relative to normal tissue (double asterisk in FIG. 13B, 132N and T in FIG. 14). Thus, for these two cases, there was a correlation between topors protein and mRNA expression. Topors relocalization enhances sumoylation of Top1 and other proteins based on the model of the present invention (See FIG. 15), which leads to Top1 relocalization and ultimately, DNA repair.

### Example 8:

### Topors mRNA is widely expressed in human tissue

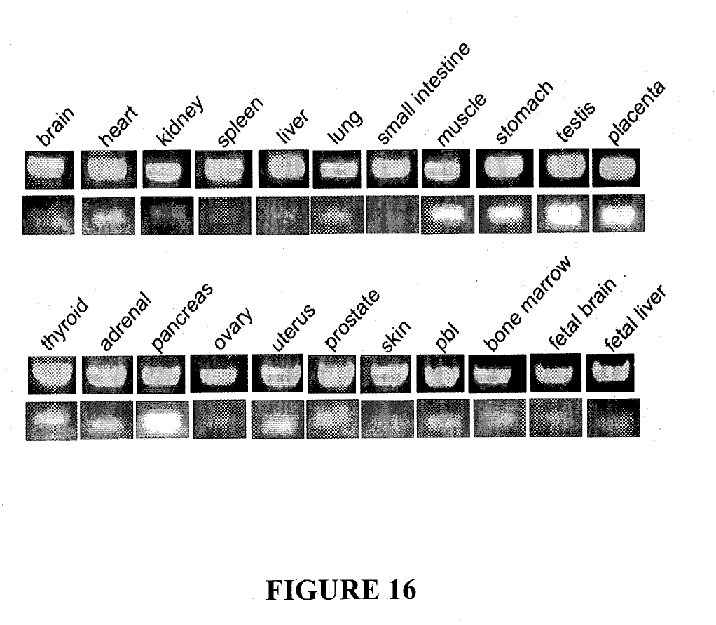

Previous studies suggested that topors mRNA was expressed at relatively low levels in human U-937 leukemia cells (Zahler et al.). To gain insight into topors biology, topors mRNA expression in human tissues using an (α-actin-normalized cDNA panel was analyzed. The results indicate that topors mRNA expression is detectable in most human tissues, with relatively high expression observed in the testis, placenta, and pancreas (FIG. 16). In FIG. 16, human tissue cDNAs normalized for β-actin mRNA expression were obtained from Origene Technologies and analyzed by PCR using topors (lower panel) and α-actin (upper panel) primers. PCR products were visualized by ethidium bromide staining of agarose gels. Serial dilutions over a 4-log range were used to establish that the resulting band intensities were linear with respect to input cDNA. Results obtained with undiluted cDNA are shown for the topors primers and for a 1:10 dilution for the actin primers.

### Example 9:

### Forced Expression of Topors Inhibits Thymidine Incorporation in Cancer Cells

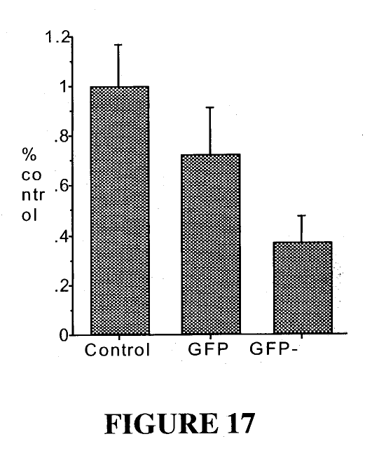

To further investigate the relative lack of topors expression in cancer tissues and cell lines, the effects of overexpression of topors on the proliferation of HeLa cells was analyzed using transient transfections and a thymidine incorporation assay. Exponentially growing HeLa cells were transfected with 5 μg of either the pEGFP (GFP) or pEGFP-topors (GFP-topors) plasmid using a lipofectamine-based method. Twelve hours after transfection, 1 μCi/ml ³H-thymidine and 150 nM cold thymidine were added to the media. After an additional 24 h, the cells were washed 4 times with ice cold PBS and fixed in methanol for 30 min at 4° C. After removal of methanol and drying, the cells were solubilized in a solution of 0.25% NaOH and 0.25% SDS. After neutralization with 1N HCl, radioactivity was quantitated by scintillation counting. Results are shown as mean and standard deviations of triplicate samples and are expressed relative to mean disintegrations per minute obtained with non-transfected HeLa cells analyzed concurrently (Control) (See FIG. 17). The difference between the mean GFP value (72%) and the mean GFP-topors value (36%) is statistically significant (p<0.05, unpaired two-way t-test).

The results indicate that transfection with pEGFP-topors significantly decreases thymidine incorporation compared to transfection with pEGFP. It is possible that the results of this experiment underestimate the anti-proliferative effects of topors overexpression, since we routinely observe about 50% of HeLa cells expressing GFP-topors after transfection with the pEGFP-topors plasmid.

### Materials and Methods

### Production of polyclonal antibody recognizing topors

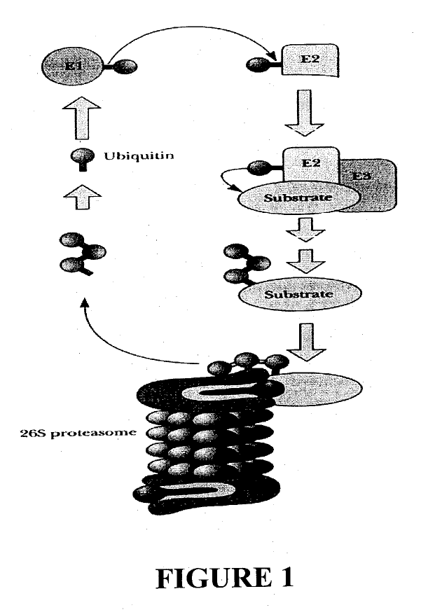

The human topors cDNA was cloned into an inducible yeast expression vector generating a GST-topors fusion protein. Recombinant topors protein was purified using glutathione sepharose beads. A polyclonal antibody was generated in rabbits using the recombinant protein. Western blot analyses indicate that the antibody recognizes recombinant GFP-topors in HeLa lysates, and an appropriately migrating band (based upon GFP-topors migration) in normal colon lysates (See FIG. 1). The predicted molecular weight of the GFP-topors is 146 kDa and for the endogenous topors is 119 kDa. However, these proteins migrated at approximately at 170 and 135 respectively. This result is consistent with the slower migration observed for other RS-rich proteins (Zahler et al., 1993).

### Loss of topors protein expression in tumor tissues

For these studies, normal and tumor specimens were obtained from the Tissue Retrieval Core of the Cancer Institute of New Jersey. The tissues were cut into small pieces, homogenized, and lysed in radioimmunoprecipitation (RIPA) buffer, then were subjected to western blot analysis using a polyclonal antibody described in the preceding section. After analyzing for the presence of topors, the same blots were stripped and reprobed with an actin antibody to control for equal protein extraction and loading.

### Expression of topors mRNA levels is decreased in tumor compared to normal tissues

Total RNA was isolated from tumor and normal tissues by a method described previously (8). A 691-bp topors fragment was generated by RT-PCR using a forward primer (5′-CGAGCACCAGCACGATAAAGAGTTCGTC-3′) [SEQ ID NO: 2] (topors RT down 2) and a reverse primer (5′-TCCTGCCGACACCGACCTAGCTTTC-3′)) [SEQ ID NO: 3]. The PCR was performed using following cycles, 50° C. for 1 h, 94° C. for 5 min, followed by 30 cycles of 94° C. for 30 sec, 65° C. for 30 sec. 68° C. for 1 min followed by 68° C. for 2 min. As a control for mRNA quantities, G3PDH primers were included in the reaction using forward primer (5′-TGAAGGTCGGAGTCAACGGATTTGGT-3′)) [SEQ ID NO: 4] and a reverse primer (5′-CATGTGGGCCATGAGGTCCACCAC-3′)) [SEQ ID NO: 5].

### Topors catalyzes SUMO conjugation to Top1

In vitro sumoylation assays were carried out as follows: Thirty microliter reactions, containing, 50 mM HEPES, pH 8.0, 5 mM MgCl₂, 15 mM ZnCl₂, 4 mM ATP, 200 ng SUMO-1, 30 ng Ubc9, 100 ng SAE1/SAE2, 1p,g His-Top1, and 1˜tg GST-topors or GST were carried out at 30° C. for 2 hours. All components of the sumoylation assay are recombinant proteins expressed in bacteria. Following the reaction the reaction mixture was resolved by SDS-PAGE and transferred to a nitrocellulose membrane and western blotted using anti-Top1 or anti-SUMO-1 antibodies.

1. A method of treating a subject having a disease or condition associated with uncontrolled cellular proliferation, comprising administering to the subject an amount of topors effective to inhibit the uncontrolled proliferation.

2. The method of claim 1, wherein the disease is cancer.

3. The method of claim 1, wherein the topors acts as a tumor suppressor by modifying or stimulating the DNA repair process by binding to Top 1.

4. The method of claim 1, wherein the topors acts as a tumor suppressor by functioning as an E3 SUMO ligase for Top1 and/or an E3 ubiquitin ligase.

5. The method of claim 1, wherein the topors acts as a tumor suppressor by activating sumoylation of p53.

6. The method of claim 1, wherein the topors is administered in conjunction with a pharmaceutically acceptable carrier.

7. The method of claim 1, wherein topors is administered by direct injection into the cells exhibiting uncontrolled cellular proliferation.

8. The method of claim 1, wherein the topors gene is administered to uncontrolled proliferating cells of the subject via a vector that expresses topors in the subject.

9. The method of claim 8, wherein the vector is a viral vector.

10. The method of claim 8, wherein the vector is a nonviral vector.

11. The method of claim 8, wherein expression of topors in the vector is controlled by a inducible promoter that is specifically directed to topors.

12. The method of claim 8, wherein expression of topors in the vector is controlled by cell and/or tissue specific cell regulatory sequences.

13. A purified antibody that specifically binds to a topors protein.

14. The purified antibody of claim 13, wherein, when the antibody binds to topors, topors is prevented from binding to top 1, p53, or any other receptor.

15. The purified antibody of claim 13, wherein the antibody is made by immunizing a non-human animal with an immunogenic fragment of topors.

16. The purified antibody of claim 13, wherein the antibody is made by generating a hybridoma cell that produces a monoclonal antibody under specific conditions for topors and culturing the cell under conditions that permit production of the monoclonal antibody.

17. A method of using the purified antibody of claim 13 to treat diseases associated with reduced vascularization and/or uncontrolled inflammation, comprising administering the topors antibody to a subject so that topors is inhibited and cellular stress is reduced.

18. A kit for screening for the presence of or susceptibility to cancer in a subject, comprising topors antibody, wherein the existence of physiologically normal levels of topors is a negative prognostic indicator of cancer and physiologically low levels of topors or the absence of topors is a positive prognostic indicator of cancer.

19. The kit of claim 18, wherein the antibody is detectably labeled.

20. A method of reducing or eliminating uncontrolled cellular proliferation of cancerous cells, comprising administering to the cells an amount of topors effective to inhibit the uncontrolled proliferation.
